(19) United States
(12) Patent Application Publication
(10) Pub. No.: US 2022/0354651 A1
(43) Pub. Date: Nov. 10, 2022

(54) BONE IMPLANT HAVING COATED POROUS STRUCTURE

(71) Applicant: WALDEMAR LINK GMBH & CO. KG, Hamburg (DE)

(72) Inventors: Helmut D. LINK, Hamburg (DE); Nicolas KONIG, Nordersted (DE)

(73) Assignee: WALDEMAR LINK GMBH & CO. KG, Hamburg (DE)

(21) Appl. No.: 17/753,437

(22) PCT Filed: Sep. 3, 2020
(86) PCT No.: PCT/EP2020/074535

§ 371 Date: Jul. 27, 2022

(30) Foreign Application Priority Data
Sep. 5, 2019 (EP) 19195580.6

(51) Int. Cl. A61F 2/30; B33Y 80/00; B33Y 40/20

(52) U.S. Cl. CPC A61F 2/30767; B33Y 80/00; B33Y 40/20; A61F 2002/3092; A61F 2002/3093; A61F 2002/30971; A61F 2310/00796; A61F 2002/30985; A61F 2002/3097; B33Y 10/00

(57) ABSTRACT

The invention relates to a bone implant, comprising a main body, which has, in its outer region, an open-cell porous lattice structure, which is formed from a plurality of regularly arranged elementary cells, the elementary cells being in the form of an assembled structure and each being composed of an interior and of a plurality of interconnected bars surrounding the interior. The porous lattice structure is provided with a bone-growth-promoting coating comprising calcium phosphate, the calcium phosphate coating having a hydroxylapatite proportion forming a pore inner coating extending into the depth of the porous lattice structure.

The invention relates to a bone implant having a main body which has an open-cell, porous lattice structure in its outer region, which lattice structure has a coating which promotes bone growth, comprising calcium phosphate.

In the case of implants, in particular endoprostheses and augmentations to be implanted in or on bones, a physiologically favorable and stable connection between implant and bone is particularly important. Furthermore, the connection is preferably established as quickly as possible in order to achieve rapid remobilization of the patient. For this purpose, it is known to provide the implant with a coating which promotes bone growth. This coating promotes the growth of bone cells and thus accelerates the growth of the implant into and/or onto the bone. In this case, it is essentially irrelevant whether the coating is applied to smooth surfaces or structured surfaces. In both cases, it fulfills its purpose.

Various materials are known for the coating. What they have in common is that they are bioactive, and in particular have properties that promote bone growth. Depending on the material, they can be applied in various ways, for example by plasma spraying, sputtering, or immersion baths. Calcium phosphate (CaP) is a material that has been known since the 1990s, and has favorable properties that promote bone growth. It has become established in implantology, in particular for the clinical application of thin and soluble coatings.

Implants have recently become known that are produced by additive methods (for example using 3D printing technology), with which a regular, macroporous structure can be produced. This is also known in the art as a trabecular structure. It has a favorable in-growing behavior already from the start. This structure has only benefitted to a limited extent from a coating with the well-known calcium phosphate. A special embodiment has become known in which calcium phosphate, with a calcium/phosphate ratio of 1.1, is used as the coating, including a brushite fraction of at least 70% and a hydroxyapatite content of up to 30%.

The invention is based on the object of creating a bone implant of the type mentioned at the outset, which has improved ingrowth behavior.

The solution according to the invention lies in the features of the independent claims. Advantageous refinements are the subject matter of the dependent claims.

According to the invention, in a bone implant having a main body which has an open-cell porous lattice structure in its outer region, which lattice structure is formed from a plurality of regularly arranged unit cells, wherein the unit cells are designed as an assembled structure and are each constructed from an interior space and a plurality of interconnected bars surrounding the interior space, wherein the porous lattice structure is provided with a coating comprising calcium phosphate, which promotes bone growth, the calcium phosphate coating has a hydroxyapatite content of at most 1 wt. %, preferably less than 1 wt. %, and forms an inner pore coating which extends into the depth of the porous lattice structure.

Some of the terms used are explained below:

An “open-cell” porous lattice structure is understood to mean that the pores are not isolated and separated; rather, the individual pores are connected to each other. This results overall in an open cell structure, with the pores being formed in the individual cells.

An “assembled structure” is understood to mean that the structure is produced additively. Various processes for additive manufacturing can be considered in this case. 3D printing processes such as electron beam melting or selective laser melting are particularly suitable.

Penetrating into the depth of the porous lattice structure is understood in this context to mean that the inner coating of the pores not only extends into the pores lying on the surface, but also covers the pores lying deeper in the material (that is, far from the surface), in particular any pores positioned several layers (of pores) away from the surface in the material.

“Wurtzite structure” means a structure modeled after the wurtzite crystal form (similar to the manner in which a diamond structure is used to mean a structure modeled after the diamond crystal form).

The core of the invention is the idea of modifying and improving the known calcium phosphate coating in such a way that it is practically free of hydroxyapatite (maximum 1 wt. %, and preferably even less than 1 wt. %). Calcium phosphate coatings known from the prior art typically have a hydroxyapatite content of as much as 20 wt. % or more, as does the special embodiment mentioned at the outset. Since, according to the invention, practically no hydroxyapatite is contained in the calcium phosphate for the coating, a higher solubility can be achieved, as the invention has recognized. The invention uses this advantage in a second step to also provide the deeper parts of the cavities created by the unit cells with the coating, thereby taking advantage of the high solubility to a greater extent.

The invention thus creates a special variant of the coating, and therefore breaks away from the previously prevailing idea of a coating on the surface of the implant that is relatively easy to apply and adheres well. Due to the extremely low content of hydroxyapatite or the absence thereof, the invention gives up the inherently good adhesion of the calcium phosphate material usually used for implant coatings. At first glance, this may seem absurd, but the invention has recognized that a decisive advantage can be realized from the apparent disadvantage of reduced adhesive strength. This opens up the possibility that this calcium phosphate, which adheres poorly in and of itself, can be brought deeper into the open-cell structure, and thus the bone growth-promoting properties can also be utilized in the depth of the open-cell structure. Surprisingly, this results in a decidedly positive effect, specifically in relation to faster bone cell growth into the implant, particularly without loss of good long-term effectiveness. This is unprecedented in the prior art.

Preferably, the calcium phosphate coating is such that it has a crystal phase comprising brushite and monetite. This combined crystal phase is at least 90 wt. %, preferably at least 95 wt. %. The extremely small fraction of hydroxyapatite according to the invention makes it possible for the combined crystal phase of brushite/monetite to make up a very high fraction, which can optionally even be 99% or more. It is preferably provided that the brushite fraction is not less than 65 wt. %. There is no lower limit for the monetite fraction. Since brushite is better degraded by the organism absorbing the bone implant, an optimum for solubility and thus for the ingrowth of bone can be ensured in particular through the minimum content of brushite.

An average thickness of the calcium phosphate coating is expediently implemented in such a manner that the interior spaces of the unit cells remain connected to each other, and is preferably between 10 and 25 μm, more preferably 15 μm+/−5 μm. The open-cell structure is thus retained despite the coating, which promotes the penetration of osteoblasts into the cells. This improves the osteointegration behavior.

Furthermore, the calcium phosphate coating preferably has a calcium/phosphate ratio in the range of 1.0 to 1.2, preferably 1.05 to 1.15. The higher phosphate content (compared to the prior art ratio of about 1.6) ensures greater solubility, which in turn promotes bone ingrowth behavior, especially together with the coating in the depth of the structure. Furthermore, the calcium phosphate coating preferably has a brushite phase which is at least 90 wt. %, preferably at least 95 wt. % (and optionally up to 100 wt. %). With this ratio, a high content of brushite in the calcium phosphate can be realized, particularly together with a minimum of, or no, hydroxyapatite, with the advantageous effects described above. In particular, it can be achieved in this way that the calcium phosphate coating is applied on all sides to the assembled structure of the unit cells—in particular to their bars. In particular, an omnidirectional (circumferential) sheathing of the pores with the calcium phosphate coating can be formed, specifically even in the case of pores that have complex or undercut structures.

Furthermore, it is expediently provided that the calcium phosphate coating is not annealed. On the one hand, this simplifies production; on the other hand, this has the advantage that an undesired reduction in the proportion of brushite, as would be brought about by tempering, can be avoided.

The unit cells are advantageously arranged in layers, wherein multiple layers are arranged one above the other, preferably as an open-cell trabecular structure. In this way, a deeper-reaching, open-cell structure can be created which also enables deeper ingrowth through osteointegration. The calcium phosphate coating is expediently also applied down to a deeper layer—preferably into all layers. A deeper layer is understood to be a layer that is not directly on the surface, but rather deeper in the material. This further improves the connection between the implant and the surrounding bone. Both short-term and long-term reliability of the attachment are thereby favored.

The open-cell porous lattice structure is expediently designed as a 3D print, preferably by means of electron beam melting (EBM) or selective laser melting (SLM). In this way, components can be manufactured efficiently and in a controlled manner from metallic materials, specifically even components that have structures with complex and numerous undercuts and cavities. The structure of the unit cells can be precisely defined in the manufacturing process. This enables a defined arrangement of the cells and of the elements forming them—in particular, their bars. In particular, these methods are suitable for producing the implant from biocompatible material, in particular metallic material, selected from a group comprising pure titanium, titanium alloys, cobalt-chrome, tantalum, stainless steel and zirconium, preferably from grade 2 or 4 titanium.

The main body advantageously consists of the same material as the open-cell porous lattice structure. As such, the same cheap, biocompatible material can also be used for the main body. Furthermore, this enables a seamless and optionally stepless transition between the open-cell porous structure and the actual main body. In addition, a more efficient production is made possible. This is especially true if the main body also has a supporting region (support region). This can expediently also have a certain porosity, but which typically is different from the open-cell lattice structure, and is preferably lower. It is particularly expedient if the support region is made of solid material. This not only results in greater mechanical strength, but also a blocking effect can be achieved, provided in the manner of a bulkhead, for example to delimit inner and outer regions or to prevent the incursion of materials such as bone cement and/or body fluids.

It is particularly expedient if the support region can be designed as a single unit together with the open-cell porous structure. This enables a particularly efficient production and a stepless transition. The latter in particular offers the advantage of minimal irritation of surrounding tissue, and thus further promotes ingrowth behavior.

The unit cells are preferably designed in a wurtzite structure. This differs from the well-known diamond structure in that the diamond structure has the same stiffness in all three dimensions of space, whereas the wurtzite structure has different stiffness in the spatial directions. This enables the stiffness behavior to be better adapted to the anatomical conditions by means of the wurtzite structure, and as a result increases the biocompatibility of the implant.

The unit cells are advantageously designed to be macroporous. In the present case, this means in particular that they form macropores with their interior spaces, the width of which is in the range between 0.4 and 2 mm, preferably 0.7 to 1.5 mm. The depth of the porous structure is expediently selected in such a manner that at least two layers of unit cells lie on top of each other. Such a macroporous open-cell structure with large, connected free spaces offers particularly favorable conditions for the cross-linked ingrowth of bones.

In contrast to the relatively large design of the pores formed by the unit cells, the coating is preferably designed to be comparatively thin. The coating expediently has a thickness of only between 10 and 20 μm. With such a thin coating, a kind of inner lining of the macropores formed by the unit cells can be achieved, specifically in such a way that the porosity, and in particular, the open-cell structure (that is, the connection between the individual free spaces), are perfectly preserved. This is particularly favorable with regard to bone ingrowth behavior, both for osteoinduction and for osteoconduction. Expediently, the ratio between the width of the free space of the unit cells on the one hand and the thickness of the coating on the other hand is selected in such a manner that the width of the free space is at least ten times, preferably between 30 times and 200 times, the thickness of the coating.

The invention also extends to a method for producing a correspondingly coated implant, having a main body which has an open-cell, porous lattice structure in its outer region, which lattice structure is formed from a plurality of regularly arranged unit cells, having the steps of: building up the regularly arranged unit cells as an assembled structure, each consisting of an interior space and a plurality of interconnected bars, the latter surrounding the interior space in such a way that the interior spaces are connected to each other, coating the porous lattice structure with a coating which promotes bone growth, comprising calcium phosphate, wherein according to the invention, upon coating, the coating is produced with a hydroxyapatite content of at most 1 wt. %, and is applied down into the deeper parts of the porous lattice structure as an inner pore coating. Expediently, the coating is carried out in such a manner that it has a crystal phase comprising brushite and monetite, and which makes up at least 90 wt. %, preferably at least 95 wt. %, wherein the fraction of brushite is not less than 65 wt. %. For a more detailed explanation, reference is made to the above description.

The coating is preferably applied to all sides of the porous lattice structure as a precipitate, preferably by means of an electrochemical process. An omnidirectional inner pore coating—that is, a sheathing of the pores with the coating which promotes bone growth—can thus be achieved. Advantageously, the electrochemical process uses a current that follows a current curve that falls back to a lower working current after an initial peak current. The inventor has recognized that this reduced current leads to an improved precipitation reaction, particularly of the combined brushite/monetite crystal phase of the calcium phosphate on the structural elements of the unit cells, particularly in the depth of the structure. Furthermore, a uniform, thin coating can be reliably achieved in this way.

Furthermore, there is preferably no subsequent annealing after the electrochemical processing. Undesirable crystal transformation can thus be avoided, so that the brushite phase of the calcium phosphate continues to retain the desired high fraction.

For further advantageous configurations and for a more detailed description, reference is made to the above explanation of the implant, which also applies correspondingly to the method.

In summary, it can be stated that improved bone ingrowth can be achieved with the coating according to the invention, as tests have shown.

The invention is explained in more detail below with reference to the attached drawing, based on advantageous embodiments. In the figures:

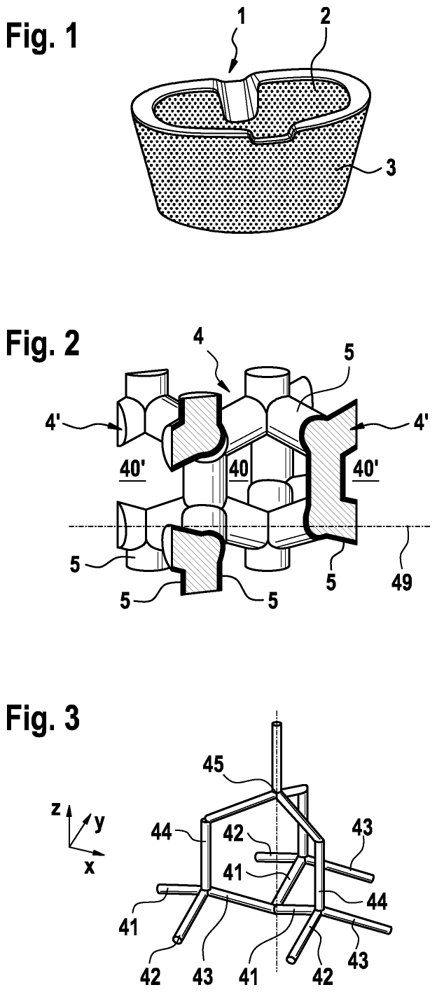

FIG. 1 is an embodiment of an implant according to an embodiment of the invention;

FIG. 2 is a detailed view of a unit cell of the porous structure of the implant according to FIG. 1;

FIG. 3 is a schematic view of the unit cells and the elements forming them;

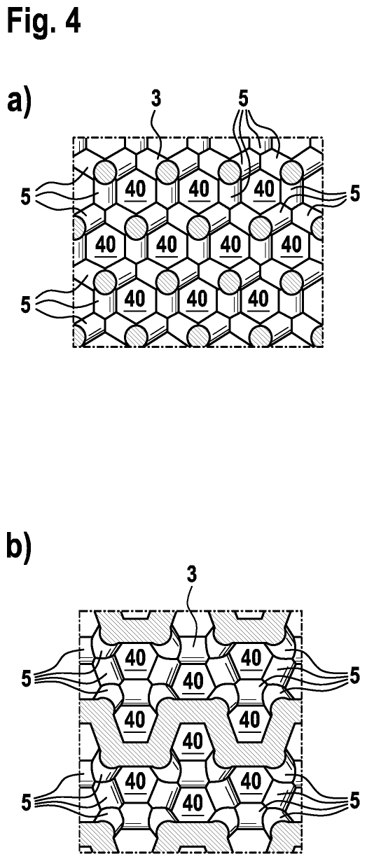

FIG. 4a, b are sectional views in two orthogonal directions of the porous structure;

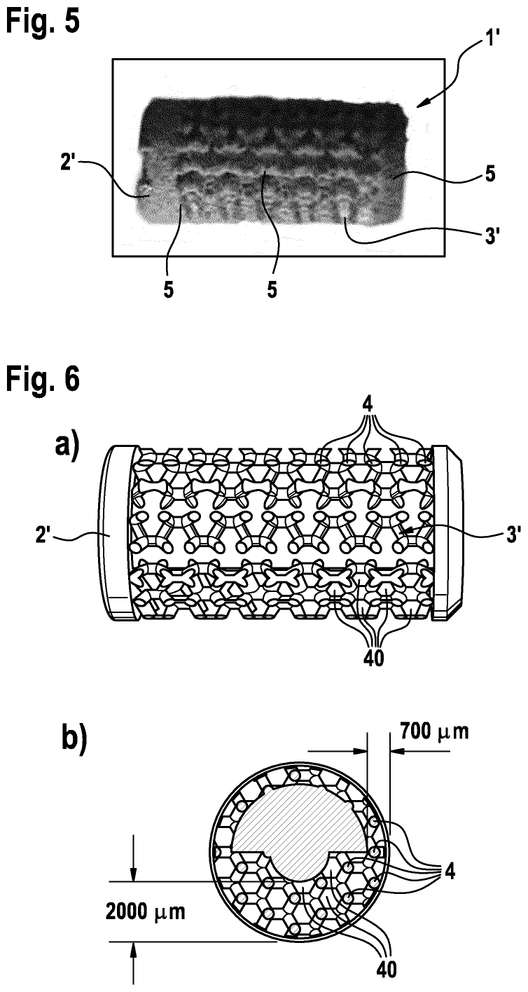

FIG. 5 is an image of an augmentation according to a second embodiment of the invention;

FIG. 6a, b are a schematic side view and front view of the augmentation according to FIG. 5;

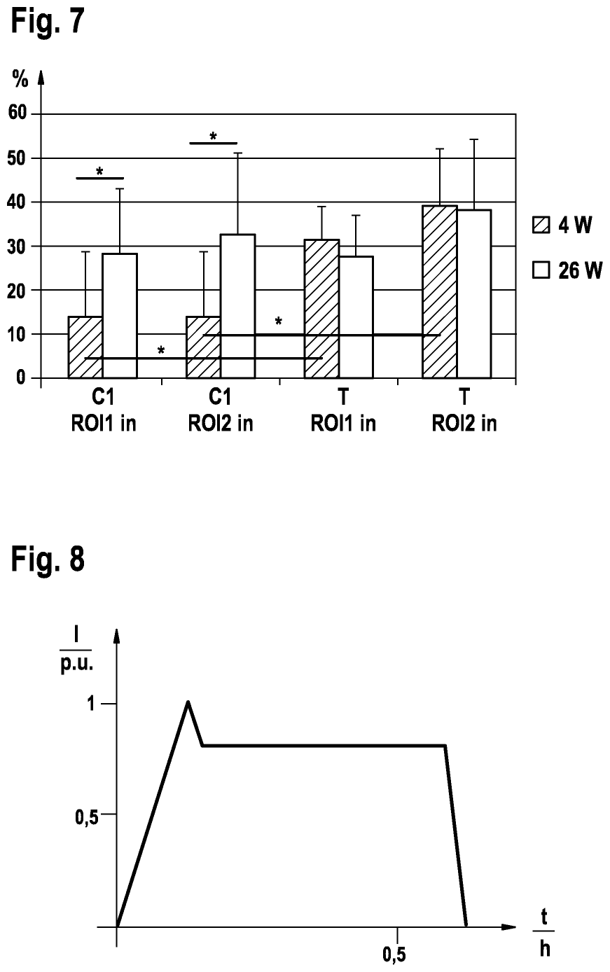

FIG. 7 is a comparative table showing bone ingrowth behavior; and

FIG. 8 is a diagram of the current curve during electrochemical coating according to the invention.

A first embodiment of an implant according to the invention is shown in FIG. 1. This implant is a cone 1 for the tibial component of a knee joint endoprosthesis (not shown).

The cone 1 forms a replacement for defective bone material at the proximal end of the tibia, so as to fill cavities which have arisen due to the absence of damaged bone material. In this way, a complete base is created upon which the tibial component of the knee joint endoprosthesis can be securely placed. For this purpose, the cone 1 is produced using the open-cell, porous lattice structure that is provided with a coating according to the invention to improve the growth of bone material into and/or onto the same. In this case, the open-cell, porous lattice structure 3 is applied to a main body 2.

Thanks in particular to the arrangement of this open-cell, porous lattice structure 3 on the outside of the cone 1, good ingrowth behavior of bone material from the surrounding tibia bone (not shown) can be achieved, resulting in the cone 1 being fixed quickly and securely in the tibia.

The porous structure 3 is formed by a plurality of regularly arranged unit cells 4. A detailed view of an unit cell 4 and its integration into the surrounding unit cells is shown in FIG. 2. The unit cell 4 has an interior space 40 which is connected to the interior space 40′ of adjacent unit cells 4′. The unit cells are arranged regularly along a layering plane 49. Advantageously, several layer levels are arranged one above the other.

The regular arrangement of the unit cells can be seen particularly well from the side views in FIG. 4a, b. These figures show isometric views along the two orthogonal axes (see axes x, y in FIG. 3) which define the layering plane 49. It can be seen that different cross-sectional views, in particular with regard to the form of the interior 40, are produced in the two directions. This is a special property of the crystal structure used, namely the wurtzite structure. It ensures that the open-cell, porous lattice structure formed in this way has different compression stiffnesses in different spatial directions, which is favorable in terms of adaptation to the anatomical conditions of the bone. It can also be seen there that adjacent inner spaces 40 are connected to each other, such that the macropores formed by the unit cells 4 with their inner spaces 40 are connected to each other in an open-celled manner (they form so-called “interconnected pores”).

The actual structure of the unit cells 4 is shown schematically in FIG. 3. In the embodiment shown, the unit cells 4 are formed from basic elements 45, which are each designed as tetrapods. It should be understood that basic elements other than tetrapods can also be provided. Each of these tetrapods has four legs 41, 42, 43, 44 designed as bars which are each connected to each other at one end, and thus form a node there. The tetrapods can be formed regularly or irregularly, with equal leg lengths or different leg lengths. Shown is a regular embodiment, where the legs are of equal length and each leg forms the same angle with each of the other legs. In the arrangement of the tetrapods in a flat layering, three legs 41, 42, 43 are arranged standing up on a plane, while the fourth leg 44 is oriented perpendicular to the plane. This fourth leg thus represents a connection to the tetrapods of a layering level arranged above it (see FIG. 3).

By choosing the number of layering levels, the depth of the open-cell porous structure can be controlled. For example, three or four or five superimposed layers can be provided (see FIG. 4a, b), but typically at least two superimposed layers are provided. A titanium alloy or pure titanium is preferably used as the material for the open-cell porous structure.

A second embodiment is shown in FIGS. 5 and 6. FIG. 5 is a photographic image. It shows a cylindrical augmentation 1′, such as can also be used for filling bone defects, or optionally also for the purpose of fusing neighboring bone elements, in particular vertebral bodies. It has a substantially sleeve-shaped main body 2′, which is generally cylindrical in shape. The main body 2′ is provided with the open-cell, porous lattice structure 3′ on its shell surface. As can be seen particularly well in the schematic view in FIG. 6a, b, it is also formed from unit cells 4 with their interior spaces 40 connected to each other, the unit cells 4 in turn consisting of tetrapods as basic elements 45.

As can be seen particularly well from the photographic image in FIG. 5, the open-cell, porous lattice structure 3′ formed by the unit cells 4 is provided with a coating 5 that appears somewhat rough in the image. The coating 5 is applied over the surface of the open-cell porous lattice structure 3′ and the two end regions of the main body 2′, and further also in the depth of the structure 3′ in the interior spaces 40 of the unit cells 4.

Exemplary dimensions for the length and width of the cylindrical sleeve-like main body 2′ are 12 mm in length and 6 mm in diameter as width. The inner spaces 40 of the unit cells 4 forming the open-cell porous structure 3′ have a width of approximately 700 μm, and the depth of the open-cell porous structure 3′ extends over approximately 2000 μm. Viewed in unit cells 4, this results in a depth of almost three layers of unit cells 4.

The coating 5 has a combined crystal phase of brushite and monetite with a fraction of 95 wt. %, the fraction of brushite being at least 65 wt. %. Furthermore, the coating 5 completely sheathes the unit cells 4 with their cavities 40, not only in the uppermost layer but also in the layers below.

According to the invention, this results in significantly improved ingrowth of bone material during the process of osteointegration and osteoconduction. Results for a comparison experiment with a comparison implant that has an open-cell porous structure of the same shape, but without a coating 5 according to the invention, are shown in FIG. 7. In the figure, a quantitative histomorphometric analysis is shown; the bone/implant contact ratio expressed as a percentage is plotted along the Y-axis for two different regions (ROI1 and ROI2). The two pairs of columns on the left represent the comparison implant (“C1”), and the two pairs of columns on the right represent the tested implant (“T”) according to the invention. The left column in each pair of columns shows the short-term ingrowth (measured at 4 weeks), and the right column in each pair of columns shows the long-term ingrowth (measured at 26 weeks). One can clearly see that with the implant (“T”) according to the invention, excellent ingrowth of bone material is already achieved after 4 weeks, with the comparative example only achieving a similar value after a good six times as long, namely after 26 weeks. This impressively demonstrates the bone growth-promoting property of the coating according to the invention.

An electrochemical process is expediently used for the coating. The profile of the current during the electrochemical coating is shown in FIG. 8.

It can be seen that a high peak current is initially set, which is then reduced to a lower working current. With this current profile, a particularly good precipitation reaction of the calcium phosphate, which is particularly suitable for the thin and uniform coating, can be achieved, with the combined brushite/monetite phase being formed with its high proportion of 95%.

1. A bone implant, comprising a main body with an open-cell porous lattice structure in its outer region, said lattice structure comprising a plurality of regularly arranged unit cells, wherein the unit cells are an assembled structure and are constructed from an interior space and a plurality of interconnected bars surrounding the interior space,
wherein the porous lattice structure is covered with a coating which promotes bone growth, comprising calcium phosphate,
characterized in that
the calcium phosphate coating has a hydroxyapatite content of less than or equal to 1 wt. %, and extends into the porous lattice structure.

2. The bone implant according to claim 1, wherein the calcium phosphate coating has a crystal phase which comprises brushite and monetite, and which is at least 90 wt. % wherein the brushite fraction is not less than is 65 wt. %.

3. The bone implant according to claim 1, wherein the calcium phosphate coating has a calcium/phosphate ratio in the range from 1.0 to 1.2.

4. The bone implant according to claim 1, wherein the interior spaces of the unit cells is between 10 and 25 μm.

5. The bone implant according to claim 1, wherein the calcium phosphate coating is unannealed.

6. The bone implant according to claim 1, wherein the calcium phosphate coating covers all sides of the assembled structure of the unit cells.

7. The bone implant according to claim 1, wherein the unit cells are arranged in layers to form an open-cell trabecular structure, and the unit cells are in a wurtzite structure.

8. The bone implant according to claim 1, wherein the open-cell porous lattice structure is a 3D printed structure, printed by means of electron beam melting (EBM) or selective laser melting (SLM).

9. The bone implant according to claim 1, wherein the main body is made of the same material as the open-cell porous lattice structure.

10. The bone implant according to claim 1, wherein the main body has a supporting region, said supporting region having a lower porosity than the porosity of the open-cell porous lattice structure.

11. The bone implant according to claim 1, wherein the inner spaces of the unit cells form macropores, the width of which is at least ten times the thickness of the coating (5), or the width of the pores is in the range between 0.4 and 2 mm and the coating has a thickness between 10 and 20 microns.

12. A method for producing a coated bone implant, having a main body which has an open-cell, porous lattice structure in its outer region, which lattice structure is formed from a plurality of regularly arranged unit cells, having the steps of:
building up the regularly arranged unit cells as an assembled structure, each consisting of an interior space and a plurality of interconnected bars surrounding the interior space in such a way that the interior spaces are connected to each other,
coating the porous lattice structure with a coating which promotes bone growth, comprising calcium phosphate, wherein
the coating is produced with a hydroxyapatite content of less than or equal to 1 wt. %, and is applied into the porous lattice structure as an inner pore coating.

13. The method according to claim 12, wherein the coating has a crystal phase which comprises brushite and monetite, and which is at least 90 wt. %, and the fraction of brushite is not less than 65 wt. %.

14. The method according to claim 12, wherein the coating is applied to all sides of the porous lattice structure by an electrochemical method.

15. The method according to claim 14, wherein a current is used for the electrochemical method, which follows a current curve which, after an initial peak current, falls back to a lower working current.

16. The bone implant according to claim 10, wherein the main body has a solid supporting region and the open-call porous lattice structure are designed as a single unit.
